(19) United States
(12) Patent Application Publication
(10) Pub. No.: US 2022/0099686 A1
(43) Pub. Date: Mar. 31, 2022

(54) BRAIN-DERIVED VESICLE-SPECIFIC MARKER AND BRAIN DISEASE DIAGNOSTIC METHOD USING SAME

(71) Applicant: CHA UNIVERSITY INDUSTRY-ACADEMIC COOPERATION FOUNDATION, Pocheon-si (KR)

(72) Inventor: Ji Sook MOON, Seoul (KR)

(73) Assignee: CHA UNIVERSITY INDUSTRY-ACADEMIC COOPERATION FOUNDATION, Pocheon-si (KR)

(21) Appl. No.: 17/426,936

(22) PCT Filed: Nov. 29, 2019
(86) PCT No.: PCT/KR2019/016777

§ 371 Date: Jul. 29, 2021

(30) Foreign Application Priority Data
Nov. 30, 2018 (KR) 10-2018-0152714

(51) Int. Cl. G01N 33/68

(52) U.S. Cl. CPC G01N 33/6896

(57) ABSTRACT

Provided are a brain-derived vesicle-specific marker and a brain disease diagnostic method using the same. According to one aspect, by using a marker which specifically expresses in brain-derived vesicles, brain-derived vesicles may be isolated in a highly efficient and highly specific manner without having to collect tissue, and vesicles which specifically express in each region of the brain may be isolated. Also, a brain disease condition may be diagnosed by means of an expression profile of a marker specific to a brain region in brain-derived vesicles which have been isolated by means of the marker according to one aspect.

### TECHNICAL FIELD

The present disclosure relates to a brain-derived vesicle-specific marker and a brain disease diagnostic method.

This research was financially supported by the Ministry of Trade, Industry, and Energy (MOTIE), Korea, under the “Regional Innovation Cluster Development Program (OpenLab, P0004793)” supervised by the Korea Institute for Advancement of Technology (KIAT).

This research was supported by the Bio & Medical Technology Development Program of the National Research Foundation (NRF) funded by the Korean government (MSIT) (No. 2019M3A9H1103765).

### BACKGROUND ART

Population aging has progressed in the recent society due to increases in universal welfare and life expectancy, and thus the number of people with age-related brain diseases such as Alzheimer's disease, Parkinson's disease, stroke, brain cancer also tend to increase. However, such degenerative brain diseases, commonly referred to as dementia, are difficult to accurately diagnose at their early stages because clinical features thereof are considerably similar to each other at the early stages and appropriate treatment time is often missed due to difficulties in early diagnosis. Particularly, because the brain is an organ virtually impossible to biopsy, there is a need to develop indirect methods of obtaining results similar to the biopsy without collecting tissue to increase specificity of screening for degenerative brain diseases.

Meanwhile, exosomes are a type of extracellular vesicles released from cells and distinguished from microvesicles because they are derived from endosomes in cells. Exosomes are known to contain proteins or miRNAs necessary for intercellular communication and it has been reported that exosomes also play a role in maintaining intracellular homeostasis by releasing harmful proteins, DNA and RNA accumulated in cells to the outside. Particularly, exosomes with small sizes of 100 nm or less have been known to freely pass through blood brain barriers and thus a large amount of brain-derived exosomes secreted from the brain is contained in human blood of human for this reason.

Brain-derived vesicles, e.g., exosomes, are a factor having drawing attention in the field of brain disease diagnosis, and research has been conducted into methods of indirectly examining the condition of the brain by isolating brain-derived exosomes from blood and analyzing molecules inside the exosome. Indirect profiles of the brain using brain-derived exosomes are advantageous in that information on brain tissue may be obtained without biopsy of the brain. Although brain-derived exosomes have been conventionally isolated from blood by targeting CD171, which is an exosome surface antibody, research into brain-derived exosomes based on CD171 is disadvantageous in that results thereof may be affected by the conditions of other organs because CD171 is also expressed in significant levels in organs other than the brain.

### DESCRIPTION OF EMBODIMENTS

### Technical Problem

Under such backgrounds, the present inventors have found a novel vesicle marker more organ-specific and capable of replacing CD171 and confirmed that vesicles isolated using a method of isolating the same and the novel vesicle marker represent the disease condition of the brain, thereby completing the present disclosure.

### Solution to Problem

An aspect provides a method of isolating brain-derived vesicles from a biological sample including: obtaining a biological sample including vesicles from a subject; and analyzing an expression level of at least one selected from the group consisting of cell adhesion molecule 2 (CADM2), amyloid beta precursor like protein 1 (APLP1), EPH receptor A7 (EPHA7), cytokine receptor like factor 1 (CRLF1), and solute carrier family 6 member 3 (SLC6A3) in the biological sample.

An aspect provides a composition for isolating brain-derived vesicles, the composition including an agent for measuring an expression level of at least one selected from the group consisting of CADM2, APLP1, EPHA7, CRLF1, and SLC6A3 in a biological sample including vesicles.

An aspect provides a method of providing information for diagnosis of a degenerative brain disease including: obtaining a biological sample including vesicles from a subject; isolating brain-derived vesicles by analyzing an expression level of at least one selected from the group consisting of CADM2, APLP1, EPHA7, CRLF1, and SLC6A3 in the biological sample; and comparing the expression levels of CADM2, APLP1, EPHA7, CRLF1, and SLC6A3 measured in the brain-derived vesicles with an expression level of a control.

Another aspect provides a composition for diagnosing a degenerative brain disease, the composition including an agent for measuring an expression level of at least one selected from the group consisting of CADM2, APLP1, EPHA7, CRLF1, and SLC6A3 in a biological sample including vesicles.

### Advantageous Effects of Disclosure

According to one aspect, by using a marker which specifically expresses in brain-derived vesicles, brain-derived vesicles may be isolated in a highly efficient and highly specific manner without having to collect tissue, and vesicles which specifically express in each region of the brain may be isolated Also, a brain disease condition may be diagnosed by means of an expression profile of a marker specific to a brain region in brain-derived vesicles which have been isolated by means of the marker according to one aspect.

### BRIEF DESCRIPTION OF DRAWINGS

FIG. 1 is a result showing mRNA expression levels of Aplp1 and Cadm2, which are genes of some candidates discovered in brain, kidney, heart, spleen, and liver tissue, and conventional brain marker, L1CAM, identified by RT-PCR.

FIG. 2 is a result showing quantified strengths of RT-PCR bands in which the mRNA expression levels of Aplp1, Cadm2, and L1CAM were confirmed in brain, kidney, heart, spleen, and liver tissue (FIG. 2A: L1CAM, FIG. 2B: APLP1, and FIG. 2C: CADM2).

FIG. 3 is a result showing relative mRNA expression of Aplp1 and L1CAM in brain, heart, kidney, and liver tissue (FIG. 3A: L1CAM and FIG. 3B: APLP1).

FIG. 4 is a result showing mRNA expression levels of Aplp1, Cadm2 and L1CAM in brain, heart, kidney, spleen, and liver tissue (FIG. 4A: L1CAM, FIG. 4B: APLP1, and FIG. 4C: CADM2).

FIG. 5 is a result showing protein expression levels of Aplp1 and Cadm2, which are genes discovered as candidates in brain, heart, kidney, spleen, and liver tissue, and conventional brain marker, L1CAM (full length and cleaved form), identified by western blotting.

FIG. 6 is a result showing mRNA expression levels of Aplp1 and Cadm2 in human brain primary cell lines (hNPC), human heart cell lines (AC16); human liver cell lines (HepG2); human kidney cell lines (HEK293), and human spleen cell lines (TK6) (FIG. 6A: APLP1 and FIG. 6B: CADM2).

FIG. 7 is a result showing the presence of APLP1 and CADM2 in exosomes isolated from the embryo-derived hNPC culture (hNPC EV) and exosomes isolated from human blood (hblood EV).

FIG. 8 is a result showing results of expression levels of CD171 (L1CAM), Aplp1 and Cadm2 in exosomes collected from blood, brain, heart, kidney, spleen, and liver of mice identified using an FACS (FIG. 8A: blood, FIG. 8B: brain, FIG. 8C: heart, FIG. 8D: kidney, FIG. 8e: spleen, and FIG. 8F: liver).

FIG. 9 is a result showing expression levels of CD171 (L1CAM), Aplp1 and Cadm2 in exosomes isolated from culture solutions of embryo-derived human brain primary cell lines (hNPC), human heart cell lines (AC16), human kidney cell lines (HEK293), human spleen cell lines (TK6), and human liver cell lines (HepG2) confirmed using an FACS (FIG. 9A: hNPC, FIG. 9B: AC16, FIG. 9C: HEK293, FIG. 9D: TK6, and FIG. 9E: HepG2).

### BEST MODE

An aspect of the present disclosure provides a method of isolating brain-derived vesicles from a biological sample.

The method includes: obtaining a biological sample including vesicles from a subject; and analyzing an expression level of at least one selected from the group consisting of cell adhesion molecule 2 (CADM2), amyloid beta precursor like protein 1 (APLP1), EPH receptor A7 (EPHA7), cytokine receptor like factor 1 (CRLF1), and solute carrier family 6 member 3 (SLC6A3) in the biological sample.

The method may further include determining the biological sample as brain-derived vesicles when the expression level is higher than an expression level of a control.

Throughout the specification, the “vesicles” may include at least one selected from the group consisting of exosomes, microparticles, microvesicles, nanosomes, extracellular vesicles, and ectosomes.

The “exosomes” refer to microsomes derived from endosomes of cells and released out of the cells, having sizes of 50 to 100 nm, and containing various molecules such as DNA, RNA, proteins, and metabolites derived from the inside of the cells.

As used herein, the “brain-derived vesicles” refer to vesicles released from various cells of brain tissue. The brain-derived vesicles may be classified into vesicles secreted from various cells, such as neuron-derived vesicles, oligodendrocyte-derived vesicles, microglia-derived vesicles, and astrocyte-derived vesicles.

The brain-derived vesicles, e.g., exosomes, are characterized in that they are easily incorporated into the blood due to their small sizes of 50 to 100 nm. Brain-derived exosomes may be isolated by analyzing expression levels of brain-derived exosome-specific marker molecules in the blood brain-derived exosomes incorporated into blood.

The brain-derived vesicles may have an increased level of CADM2, APLP1, EPHA7, CRLF1, SLC6A3, or any combination thereof compared to vesicles derived from other organs. That is, the CADM2, APLP1, EPHA7, CRLF1, SLC6A3, or any combination thereof may be a brain-specific, brain region-specific marker.

The CADM2, APLP1, EPHA7, CRLF1, SLC6A3, or any combination thereof may be a protein present in membranes of vesicles.

The analysis may be performed by an agent including an antibody or antigen-binding fragment specifically binding to at least one selected from the group consisting of CADM2, APLP1, EPHA7, CRLF1, and SLC6A3 or a fragment thereof.

The antibody may be fragmented using conventional techniques, and fragments may be screened for utility in the same manner as described above with respect to whole antibody. For example, F(ab)2 fragments may be produced by treating the antibody with pepsin. The produced F(ab)2 fragments may be treated to reduce disulfide bonds, thereby producing Fab fragments. The antigen-binding portions may also be produced by recombinant DNA technique or by enzymatic or chemical cleavage of an intact antibody. The antigen-binding portions include Fab, Fab′, F(ab′)2, Fv, dAb and complementarity determining region (CDR) fragment, single chain antibody (scFv), single domain antibody, double specific antibody, chimeric antibody, humanized antibody, diabody, and a polypeptide including at least a portion of immunoglobulin sufficient to confer the ability of specific antigen binding to the polypeptide. In certain embodiments, the antibody further includes a label attached thereto and detectable (e.g., the label may be a radioisotope, a fluorescent compound, an enzyme, or an enzyme cofactor).

Those of ordinary skill in the art are aware of various methods and devices available in the measurement and analysis. For polypeptides or proteins contained in samples of patients, immunoassay devices and methods are often used. These devices and methods may generate a signal related to the presence or amount of an analyte of interest by using labeled molecules in various sandwiches and competitive or non-competitive assay formats. Additionally, the presence or amount of an analyte may be determined without the need for labeled molecules using certain methods and devices, such as biosensors and optical immunoassays. Although other methods (e.g., measurement of marker RNA level) are well known to those skilled in the art, immunoassay is preferably used for the measurement. The presence or amounts of markers are generally identified by detecting specific binding using an antibody specific for each marker. Any suitable immunoassay, e.g., enzyme-linked immunosorbent assay (ELISA), radioimmunoassay (RIA), competitive binding assay, and planar waveguide technique, may be used. Specific immunological binding of the antibody to a marker may be detected directly or indirectly. Direct labels include fluorescent or luminescent tags, metals, dyes, and radionuclides attached to the antibody. Indirect labels include various enzymes well known in the art, e.g., alkaline phosphatase and horseradish peroxidase.

The brain-derived vesicles may be isolated from the biological sample or concentrated by bringing the biological sample into contact with the agent under the condition that the vesicles contained in the biological sample bind to the agent to form a vesicle-agent complex; and separating the vesicles from the vesicle-agent complex to obtain a sample containing the vesicles.

The expression level may be a protein level of CADM2, APLP1, EPHA7, CRLF1, SLC6A3, or any combination thereof.

The sample may be obtained from a cell culture supernatant, whole blood, serum, plasma, ascites fluid, cerebrospinal fluid, bone marrow aspirate, bronchoalveolar lavage fluid, urine, semen, vaginal fluid, mucus, saliva, sputum, or purified lysates from a biological tissue sample, or for example, obtained from any other sources known in the art including brain tissue-containing other tissue.

When the sample is blood, it may be combined with various components after collection to preserve or prepare the sample for subsequent techniques. For example, the blood is treated with an anticoagulant, a cell fixative, a protease inhibitor, a phosphatase inhibitor, a protein, or a DNA, or a RNA preservative after collection. For example, blood is collected via venipuncture using vacuum collection tubes containing an anticoagulant such as EDTA or heparin. Blood may also be collected using a heparin-coated syringe and hypodermic needle. Blood may also be combined with components that will be useful for cell culture. For example, blood may be combined with a cell culture medium or a supplemented cell culture medium (e.g., cytokines). Also, when the sample is blood, it is advantageous that the brain-derived vesicles may be isolated by using vesicles freely passing through the blood brain barriers without collecting brain tissue.

Another aspect provides a composition for isolating brain-derived vesicles, the composition including an agent for measuring an expression level of at least one selected from the group consisting of CADM2, APLP1, EPHA7, CRLF1, and SLC6A3 in a biological sample including vesicles.

The biological sample, the vesicles, the brain-derived vesicles, and the agent for measuring expression levels are as described above.

In an embodiment, in the brain-derived vesicles, the expression level of CADM2, APLP1, EPHA7, CRLF1, or SLC6A3 is increased when compared with vesicles derived from other organs, and thus brain-derived vesicles may be isolated by measuring the expression level thereof.

Another aspect provides a kit for isolating brain-derived vesicles including an agent for measuring an expression level of at least one selected from the group consisting of CADM2, APLP1, EPHA7, CRLF1, and SLC6A3 in a biological sample including vesicles.

Various kits including different components are contemplated by the present disclosure. Generally speaking, the kit may include a means of quantifying one or more markers in a subject. The kit may include an element for collecting a biological sample, an element for quantifying at least one biomarker in the biological sample, and manuals for use of the elements of the kit. The kit may include an element for concentrating or isolating vesicles from the biological sample. In an additional aspect, the element for concentrating or isolating vesicles includes a reagent required for concentrating or isolating the vesicles from the biological sample. The kit may include an element for quantifying the amount of the biomarker and the means for quantifying the amount of the biomarker may include a reagent required for detecting the amount of the biomarker.

Another aspect provides a method of providing information for diagnosis or prognosis of a degenerative brain disease.

The method includes: obtaining a biological sample including vesicles from a subject; isolating brain-derived vesicles by analyzing an expression level of at least one selected from the group consisting of CADM2, APLP1, EPHA7, CRLF1, and SLC6A3 in the biological sample; and comparing the expression level of CADM2, APLP1, EPHA7, CRLF1, and SLC6A3 measured in the brain-derived vesicles with an expression level of a control.

The biological sample, the vesicles, the brain-derived vesicles, and measurement of the expression level are as described above.

The brain-derived vesicle-specific markers CADM2, APLP1, EPHA7, CRLF1, and/or SLC6A3 may be both brain-derived vesicle-specific markers and brain region-specific markers. That is, expression of each of the proteins may be increased in a certain region of the brain. For example, APLP1 may be expressed in the whole brain, CADM2, EPHA7, and CRLF1 may be specifically expressed in hippocampus, and SLC6A3 may be specifically expressed in the midbrain.

Because these markers are markers specific for certain regions of the brain, a disease may be diagnosed and prognosis may be made by isolating vesicles derived from a certain brain region and measuring the expression level of a disease marker, e.g., gene or protein, contained in the vesicles.

The disease marker may include at least one selected from the group consisting of amyloid beta, phosphorylated Tau, Aβ1-42, TDP-43, α-synuclein, SOD-1, FUS, FKBP51, IRS-1, phosphorylated IRS-1, CTSD, LAMP1, UBP, HSP70, NSE, NFL, CD9, CD63, CD81, and CD171.

The gene expression level of the disease marker may be measured by measuring the level of mRNA or miRNA. Those of ordinary skill in the art are aware of various methods and devices available in the detection and analysis of the disease marker. For polypeptides or proteins contained in samples of patients, immunoassay devices and methods are often used. These devices and methods may generate a signal related to the presence or amount of an analyte of interest by using labeled molecules in various sandwiches and competitive or non-competitive assay formats. Additionally, the presence or amount of an analyte may be determined without the need for labeled molecules using certain methods and devices, such as biosensors and optical immunoassays.

Although other methods (e.g., measurement of marker RNA level) are well known to those skilled in the art, the disease marker is analyzed, for example, by immunoassay. The presence or amounts of markers are generally identified by detecting specific binding using an antibody specific for each marker. Any suitable immunoassay, e.g., enzyme-linked immunosorbent assay (ELISA), radioimmunoassay (RIA), competitive binding assay, and planar waveguide technique, may be used. Specific immunological binding of an antibody to a marker may be detected directly or indirectly. Direct labels include fluorescent or luminescent tags, metals, dyes, and radionuclides attached to the antibody. Indirect labels include various enzymes well known in the art, e.g., alkaline phosphatase and horseradish peroxidase.

The use of immobilized antibodies specific to a disease marker is also contemplated by the present disclosure. The antibodies could be immobilized onto a variety of solid supports, such as, magnetic or chromatographic matrix particles, the surface of an assay place (e.g., microtiter wells), pieces of a solid substrate material (e.g., plastic, nylon, and paper), and the like. An assay strip could be prepared by coating an antibody or a plurality of antibodies in an array on a solid support. This strip could then be dipped into a test sample and then processed quickly through wash and detection steps to generate a measurable signal, such as a colored spot.

The analyses of a plurality of markers may be carried out separately or simultaneously with one test sample. Several markers may be combined into one test for efficient processing of a plurality of samples. In addition, one skilled in the art would recognize the value of testing multiple samples (for example, at successive time points) from the same subject. Such testing of serial samples will allow the identification of changes in marker levels over time. In addition to increases or decreases in marker levels, the absence of changes in marker levels may provide useful information on the disease condition that includes, but is not limited to, identification of an approximate time from onset of an event, identification of the presence and amount of salvageable tissue, appropriateness of a drug therapy, effectiveness of various therapies, and the severity of the event, identification of severity of a disease, and identification of results of a patient including risk of a future event.

The disease markers may play an important role in early detection and monitoring of neurodegenerative disorders and brain cancer. Disease markers are substances found in a bodily sample that may be typically measured. A measured amount may correlate to underlying disorder or disease pathophysiology, presence or absence of a neurodegenerative disorder, probability of a neurodegenerative disorder in the future. In patients receiving treatment for their condition, the measured amount may also correlate with responsiveness to therapy.

As used herein, the “degenerative brain disease” may be selected from the group consisting of Alzheimer's disease, Parkinson's disease, Huntington's disease, stroke, multiple system atrophy, vascular disease dementia, frontotemporal dementia (FTD), cortical basal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron disease, Guam parkinsonism-dementia complex, FTDP-17, Lytico-Bodig disease, multiple sclerosis, brain cancer, and traumatic brain injury (TBI).

In an embodiment, it was confirmed that a degenerative brain disease may be specifically diagnosed in a degenerative brain disease animal model as a result of measuring an expression level of at least one selected from the group consisting of CADM2, APLP1, EPHA7, CRLF1, and SLC6A3 in brain-derived vesicles isolated from a biological sample.

In another embodiment, the present disclosure enables a medical practitioner to diagnose or prognose one or more degenerative brain diseases in a subject. Alternatively, the present disclosure enables a medical practitioner to exclude or eliminate one or more degenerative brain diseases as a diagnostic possibility. Alternatively, the present disclosure enables a medical practitioner to identify a subject at the risk of developing a degenerative brain disease. Alternatively, the present disclosure enables a medical practitioner to predict whether a subject will develop a degenerative brain disease in the future. Also, the present disclosure enables a medical practitioner to prescribe therapeutic treatment or predict benefits from therapy in a subject having a degenerative brain disease.

The method plays an important role in early detection and monitoring of degenerative brain diseases. These disease markers are substances found in a bodily sample that may be typically measured. A measured amount may correlate to underlying disorder or disease pathophysiology, presence or absence of a degenerative brain disease, probability of a degenerative brain disease and brain cancer in the future. In patients receiving treatment for their condition, the measured amount will also correlate with responsiveness to the treatment.

### MODE OF DISCLOSURE

Hereinafter, the present disclosure will be described in more detail with reference to the following examples. However, the following examples are merely presented to exemplify the present disclosure, and the scope of the present disclosure is not limited thereto.

### EXAMPLE 1

### Discovery and Verification of Brain-Derived Exosome-Specific Marker

1.1. Selection of Novel Public Database (DB)-Based Brain-Specific Marker

To identify protein expression patterns of respective human organs, public database, The Human Protein atlas, which provides patterns of mRNA expression and protein staining of human tissue, was used. 419 proteins, Brain-enriched proteins, labeled with annotation data indicating that they are specifically expressed in human brains were selected from the DB as candidates.

Then, 214 proteins present in membranes of exosomes were selected therefrom using GENEONTOLOGY and Uniprot database of the 419 candidate proteins.

Then, the Allen brain atlas DB was used to identify regions of the brain where the 214 candidate proteins are expressed. The DB provides region-specific gene expression levels in the mouse brain via in situ hybridization. Based thereon, genes expressed in the global region of the brain and region-specific genes specifically expressed in the hippocampus and midbrain were selected.

1.2 Verification of Expression Specificity of Novel Marker Group in Brain

The proteins specifically expressed in the brain and selected from the public DB as described in 1.1. above were actually identified in mice. Expression of mRNA of the novel marker group in RNA collected from brain, liver, kidney, heart, and spleen of mice was identified by polymerase chain reaction (PCR) and expression of protein was identified by western blotting in the candidates whose brain-specific mRNA expression was confirmed based on the PCR results.

Specifically, PCR was performed as follows. 8-week-old mice were perfused with PBS and each organ (brain, kidney, heart, lung, and liver) was excised and frozen with liquid nitrogen. Each tissue was disrupted while maintaining at −80° C. RNA was extracted using Trizol, and cDNA was synthesized using 1 μg of the extracted RNA (Intron, 25082). PCR of a next candidate was performed using 2 μg of the synthesized cDNA. Information on genes used in the PCR and primers for each gene are as shown in Table 1.

TABLE 1

| | | SEQ | |
|---|---|---|---|
| | | ID | |
| Gene | Sequence | NO: | Conditions |
| Aplp1 | Forward: | 1 | 56° C., hot |
| | GCCACTGTCATTGCTGCTTC | | start kit, |
| | Reverse: | 2 | 28~30 cycle, |
| | GGGTTAGACGCCCACATAGTC | | 3 step |
| Cadm2 | Forward: | 3 | 56° C., |
| | CGATCCGATTGTCCCTTAAAGC | | normal kit, |
| | Reverse: | 4 | 28~30 cycle, |
| | AGGGCAGTTTCCACTAACTCA | | 3 step |
| L1CAM | Forward: | 5 | 58° C., |
| | AAAGGTGCAAGGGTGACATTC | | normal kit, |
| | Reverse: | 6 | 33 cycle, |
| | TCCCCACGTTCCTGTAGGT | | 3 step |

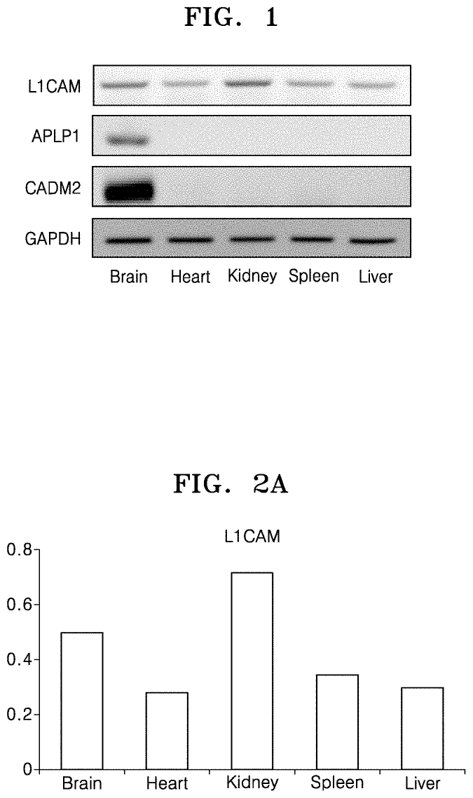

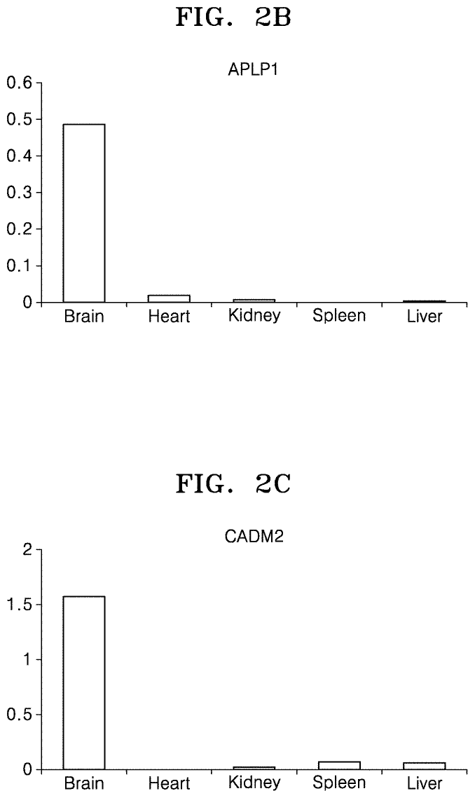

FIG. 1 is a result showing mRNA expression levels of Aplp1 and Cadm2, which are genes of some candidates discovered in brain, kidney, heart, spleen, and liver tissue, and conventional brain marker, L1CAM, identified by RT-PCR. FIG. 2 is a result showing quantified strengths of RT-PCR bands in which the mRNA expression levels of Aplp1, Cadm2, and L1CAM were confirmed in brain, kidney, heart, spleen, and liver tissue (FIG. 2A: L1CAM, FIG. 2B: APLP1, and FIG. 2C: CADM2).

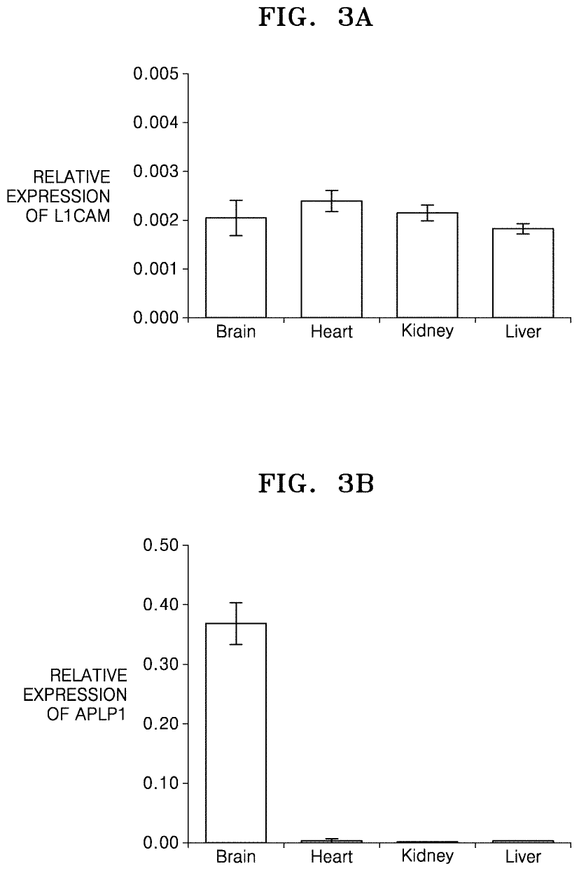

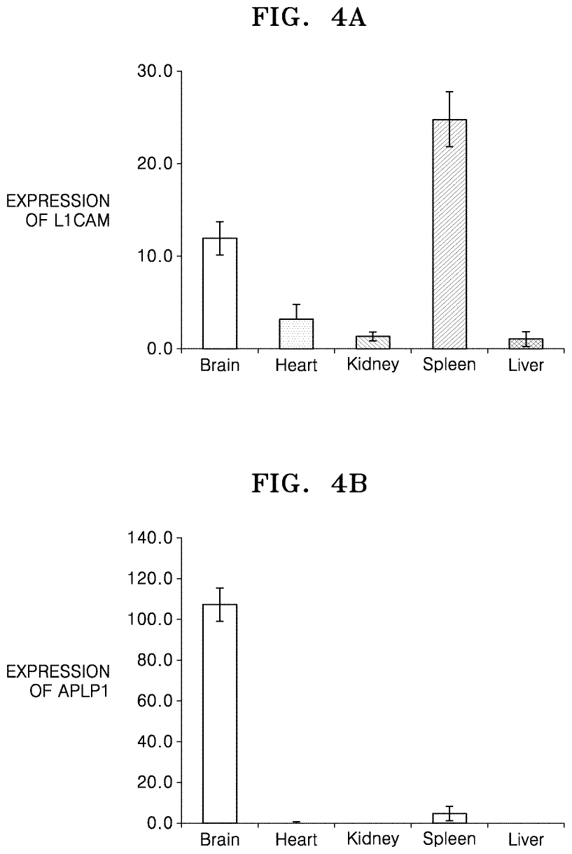

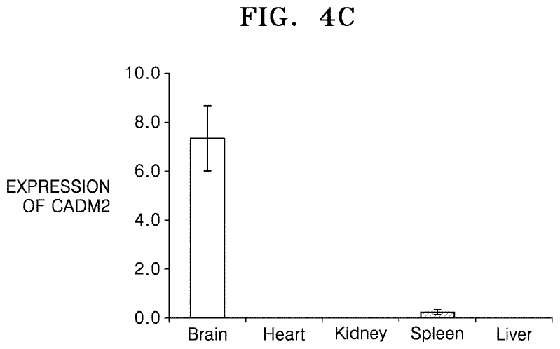

FIG. 3 is a result showing relative mRNA expression of Aplp1 and L1CAM in brain, heart, kidney, and liver tissue (FIG. 3A: L1CAM and FIG. 3B: APLP1). FIG. 4 is a result showing mRNA expression levels of Aplp1, Cadm2 and L1CAM in brain, heart, kidney, spleen, and liver tissue (FIG. 4A: L1CAM, FIG. 4B: APLP1, and FIG. 4C: CADM2).

As shown in FIGS. 1 to 4, although expression of L1CAM, which is conventionally used as a brain marker, was confirmed, it was confirmed that L1CAM is not only specifically expressed in the brain and but also in the kidney, heart, lung, and liver. On the contrary, expression of CADM2 and APLP1 was confirmed only in the brain at high levels. Therefore, it was confirmed that CADM2 and APLP1 are brain-specific markers. [0076]

To identify expression levels of CADM2 and APLP1 confirmed as brain markers, western blotting was performed. Western blotting was performed as follows. 8-week-old mice were perfused with PBS and each organ (brain, kidney, heart, spleen, and liver) was excised and frozen with liquid nitrogen. Each tissue was disrupted while maintaining at −80° C. Proteins were extracted using an RIPA buffer and quantified by a BCA assay. 15 μg of the proteins were run in a gel (60 V 1 hr and 120 V 1.5 hrs). After running, they were transferred using a membrane with 0.2 μm holes (in a cold room at 20 V, overnight (O/N)). The resultant was blocked with 10% skim milk at room temperature for 2 hours and incubated with primary antibodies diluted with 5% skim milk (anti-L1CAM antibody: ab20148, Abcam; anti-APLP1 antibody: ab192481, Abcam; and anti-CADM2 antibody: bs-8246R, Bioss Inc.) (APLP1: 1:2000, 4° C., O/N; CADM2: 1:1000, 4° C., O/N; L1CAM: 1:1000, 4° C., O/N; GAPDH: 1:10000, 4° C., O/N). The resultant was washed four times with TBST for 15 minutes. The resultant was incubated with secondary antibodies diluted with 5% skim milk (APLP1 & CADM2: Rabbit HRP 1:1000, RT, 2 hrs; L1CAM & GAPDH: Mouse HRP 1:1000, RT, 2 hrs). After incubation was completed, the resultant was developed to complete the experiment.

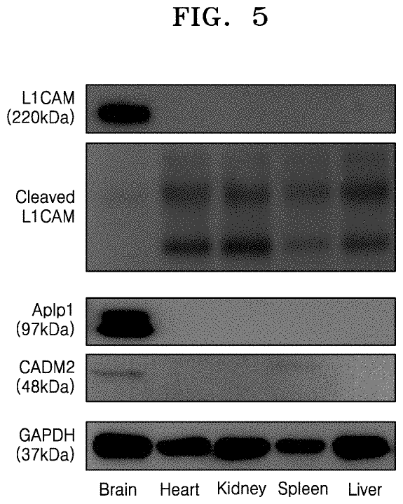

FIG. 5 is a result showing protein expression levels of Aplp1 and Cadm2, which are genes discovered as candidates in brain, heart, kidney, spleen, and liver tissue, and conventional brain marker, L1CAM (full length and cleaved form), identified by western blotting.

As shown in FIG. 5, it was confirmed that the band of the full length of L1CAM was only confirmed in the brain without being confirmed in the other tissues. Because the band of the cleaved form obtained by metalloprotease and plasm in was identified in the other tissues, it was confirmed that it was not a brain-specific marker. On the contrary, because Aplp1 and Cadm2 proteins were expressed only in the brain without being expressed in the other tissues, they were confirmed as a brain-specific markers.

1.3. Identification of Novel Marker in Brain-Derived Exosome

As well as in the mice, it was identified whether APLP1 and CADM2 were specifically expressed in human brains. To compare certain cells (human heart cell lines (AC16); human liver cell lines (HepG2); human kidney cell lines (HEK293); and human spleen cell lines (TK6)) with embryo-derived human brain primary cell lines (hNPCs) in various tissues, RNA was isolated from cell cultures and cDNA was synthesized, and then real-time polymerase chain reaction (real-time PCR) was performed.

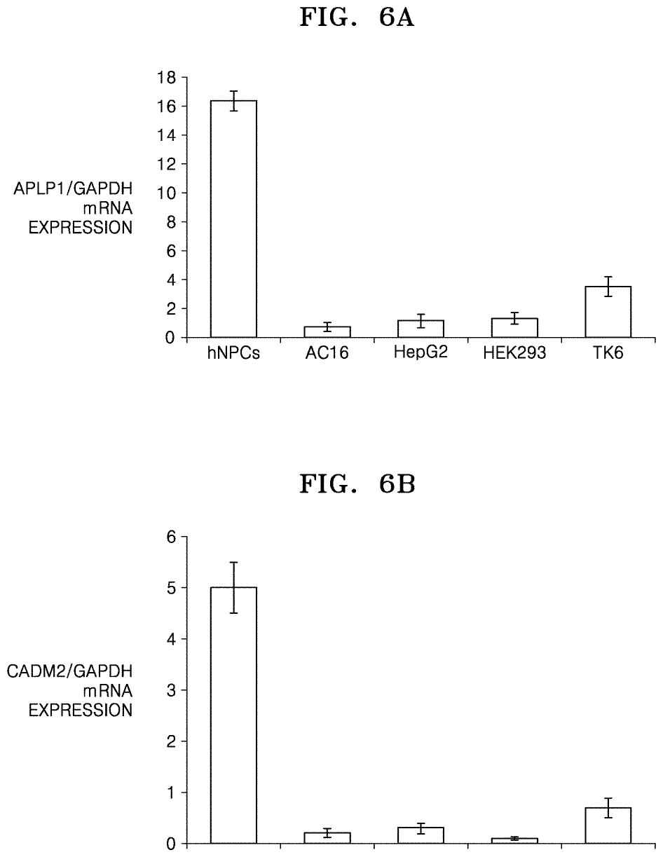

FIG. 6 is a result showing mRNA expression levels of Aplp1 and Cadm2 in human brain primary cell lines (hNPC), human heart cell lines (AC16); human liver cell lines (HepG2); human kidney cell lines (HEK293), and human spleen cell lines (TK6). As shown in FIG. 6, it was confirmed that expression levels of Aplp1 and Cadm2 were specifically high only in the cultures of the human brain primary cell lines (hNPCs)

In addition, western blotting was performed to identify whether Aplp1 and Cadm2 proteins exist in exosomes isolated from the hNPC culture and exosomes extracted from human blood.

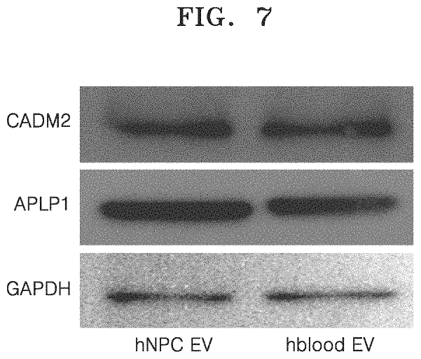

FIG. 7 is a result showing the presence of APLP1 and CADM2 in exosomes isolated from the embryo-derived hNPC culture (hNPC EV) and exosomes isolated from human blood (hblood EV). As shown in FIG. 7, it was confirmed that Aplp1 and Cadm2 were expressed at high levels in the exosomes isolated or extracted from the human brain primary cell lines and human blood.

### EXAMPLE 2

### Verification of Compatibility and Distinction Between Exosomes Positive to CADM2, APLP1, EPHA7, CRLF1, and SLC6A3 and Exosomes Positive to CD171 (CD171+ Exosome)

2.1. Identification of Compatibility of CD171+ Exosome

To identify compatibility of exosomes positive to CADM2 and APLP1, which are novel markers of brain-derived vesicles confirmed in Example 1, with exosomes positive to CD171 (L1CAM), which is a conventional brain marker, exosomes collected from mouse blood were treated with CADM2, APLP1, and CD171 respectively labeled with a fluorescent marker and examined by using a fluorescence activated cell sorter (FACS).

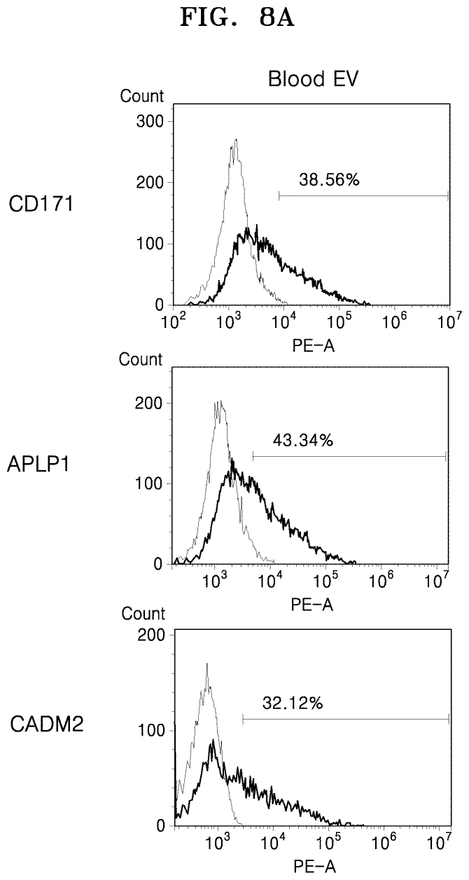

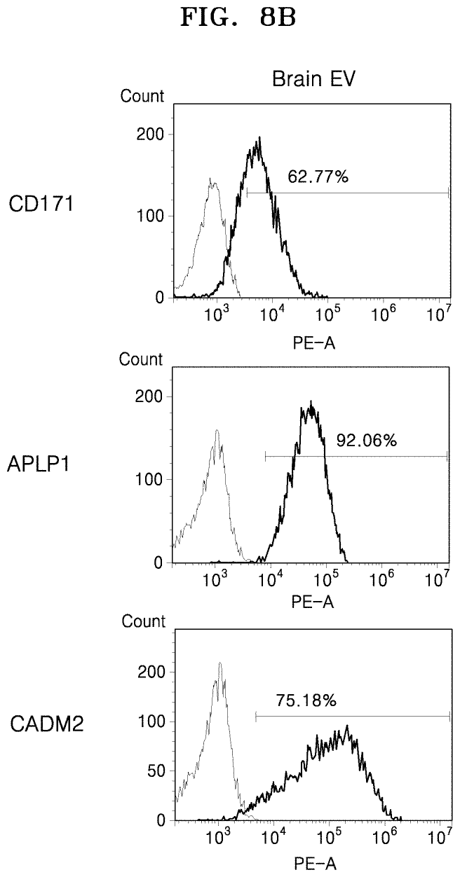

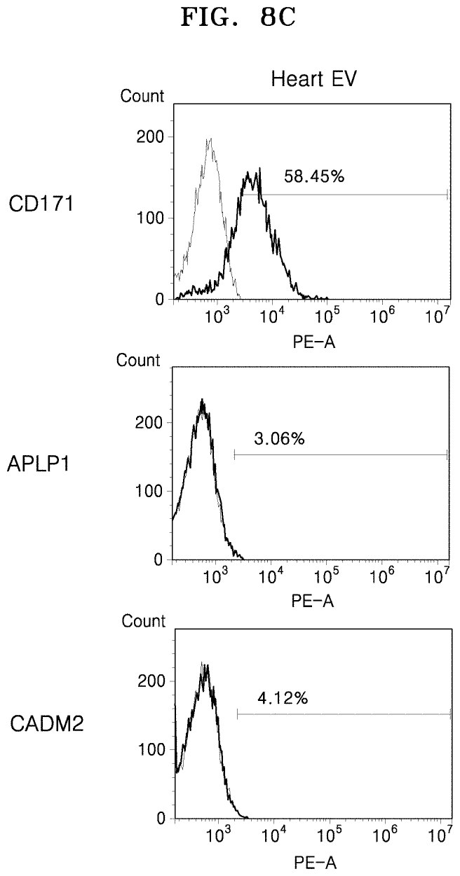

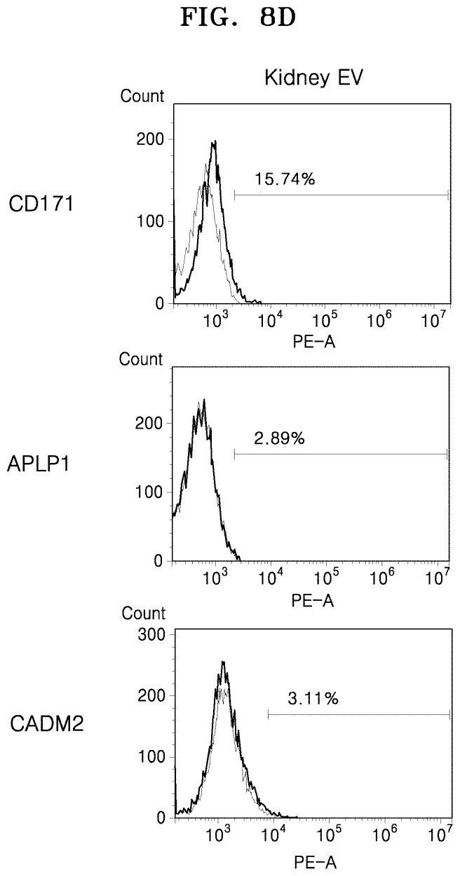

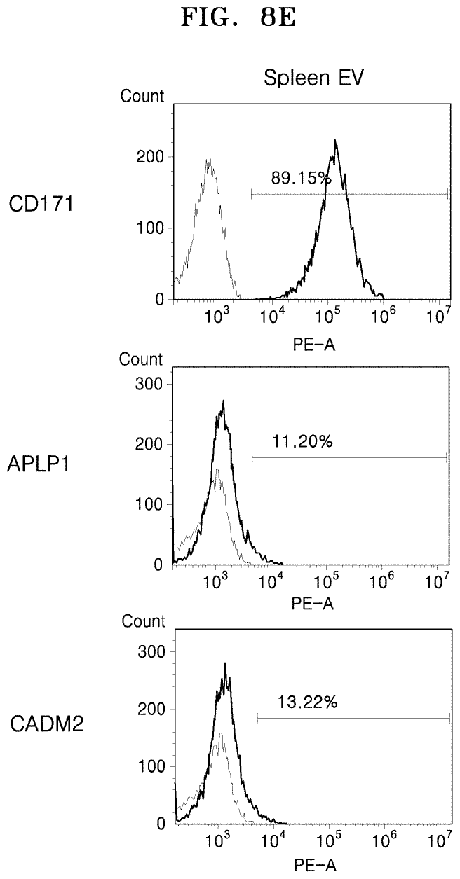

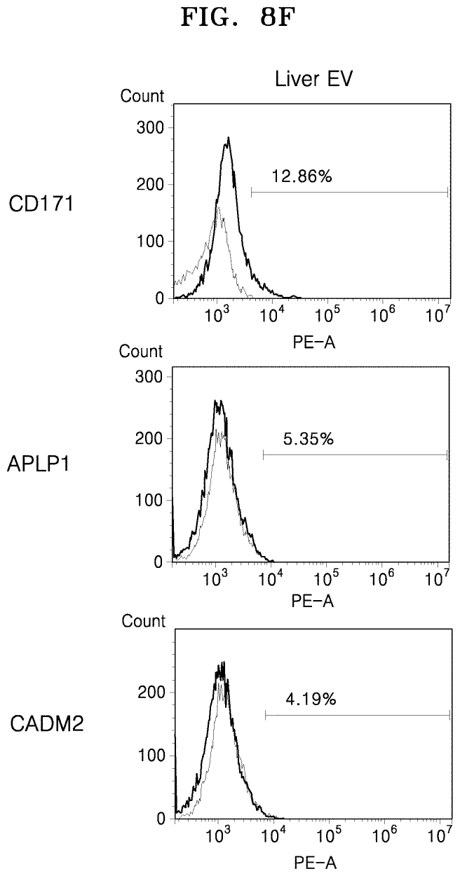

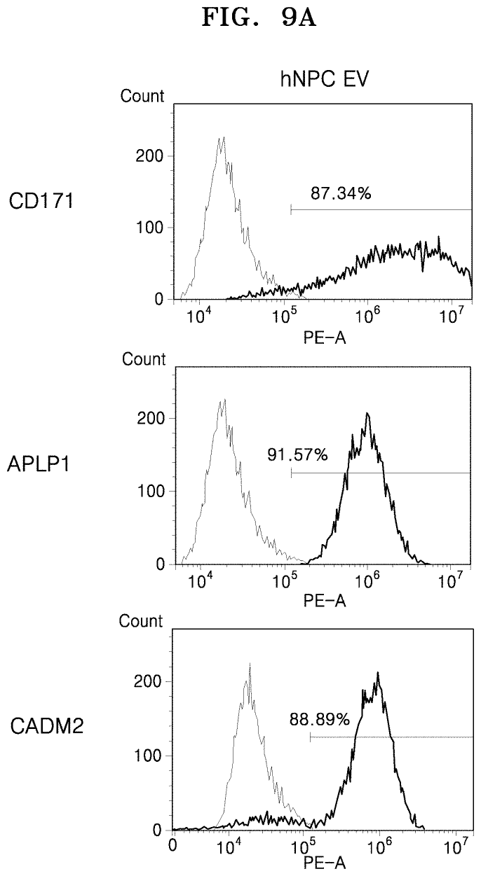

FIG. 8A is a result showing expression levels of CD171 (L1CAM), Aplp1, and Cadm2 in exosomes collected from mouse blood using an FACS. As a result, as shown in FIG. 8A, it was confirmed that the CD171+ exosomes and exosomes positive to APLP1 and CADM2 exhibited similar expression levels in the exosomes collected from the blood.

Based on the results, it was confirmed that exosomes positive to APLP1 and CADM2 have compatibility with CD171-positive exosomes.

2.2. Identification of Distinction of CD171+ Exosome

To identify distinction between the novel markers CADM2 and APLP1 and the conventional marker CD171, exosomes were isolated from sections of various organs of mice in addition to blood and fluorescence-labeled as APLP1, CADM2, and CD171, followed by examination using an FACS.

FIGS. 8B to 8F show results of expression levels of CD171 (L1CAM), Aplp1 and Cadm2 in exosomes collected from brain, heart, kidney, spleen, and liver of mice identified using an FACS. As a result, as shown in FIGS. 8B to 8F, although a large amount of CD171+ exosomes were confirmed in the exosomes isolated from the heart and spleen as well as from the brain, a specifically large amount of exosomes positive to the novel markers CADM2 and APLP1 was confirmed only in the exosomes isolated from the brain

Based on the result, it may be confirmed that APLP1 and CADM2 are distinguished from the conventional marker CD171 in that they are present in the brain-derived vesicles in specifically large amounts.

To confirm whether APLP1 and CADM2 have distinctions from the conventional brain marker CD171 in humans as well as in mice, exosomes were isolated from culture solutions obtained from various human cell lines and CD171 and APLP1 and CADM2 were fluorescence-labeled and examined using an FACS.

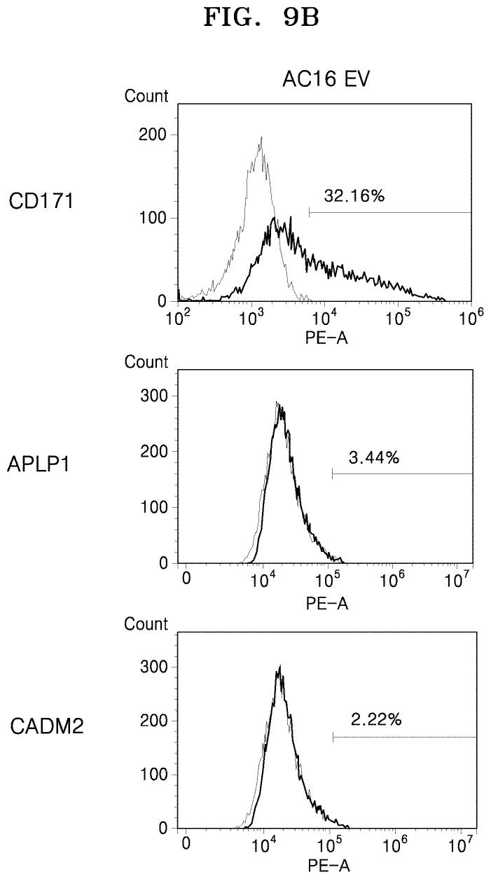

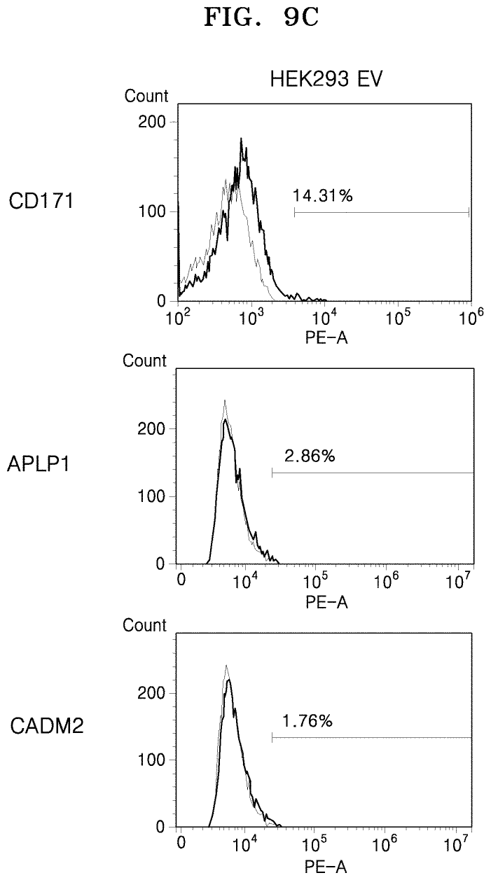

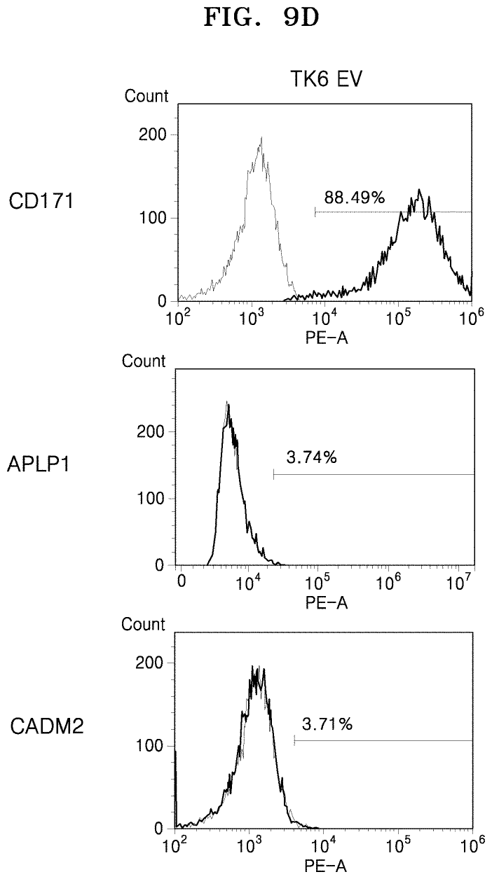

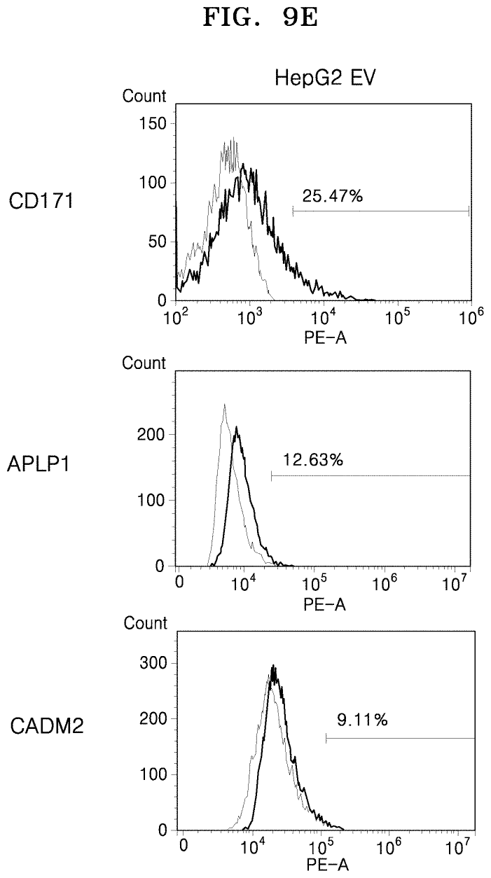

FIG. 9 is a result showing expression levels of CD171 (L1CAM), Aplp1 and Cadm2 in exosomes isolated from culture solutions of embryo-derived human brain primary cell lines (hNPC), human heart cell lines (AC16), human kidney cell lines (HEK293), human spleen cell lines (TK6), and human liver cell lines (HepG2) confirmed using an FACS. As a result, as shown in FIG. 9, while CD171+ exosomes were found abundantly in spleen cell lines, Aplp1 and Cadm2 were found specifically in high concentrations only in the human brain primary cell lines (hNPC).

Based on the results, it was confirmed that the brain-derived exosomes may be isolated from a blood sample easy to collect in a highly efficient and highly specific manner using the APLP1, CADM2, EPHA7, CRLF1, and SLC6A3 according to the present disclosure and exosomes specifically expressed in brain regions may be isolated.

1. A method of isolating brain-derived vesicles from a biological sample, the method comprising:
obtaining the biological sample including vesicles from a subject; and
analyzing an expression level of at least one selected from the group consisting of cell adhesion molecule 2 (CADM2), amyloid beta precursor like protein 1 (APLP1), EPH receptor A7 (EPHA7), cytokine receptor like factor 1 (CRLF1), and solute carrier family 6 member 3 (SLC6A3) in the biological tissue sample.

2. The method of claim 1, wherein the biological sample is a cell culture supernatant, whole blood, serum, plasma, ascites fluid, cerebrospinal fluid, bone marrow aspirate, bronchoalveolar lavage fluid, urine, semen, vaginal fluid, mucus, saliva, sputum, or purified lysates from a biological sample.

3. The method of claim 1, wherein the brain-derived vesicles are neuron-derived vesicles, oligodendrocyte-derived vesicles, microglia-derived vesicles, astrocyte-derived vesicles, or a combination thereof.

4. The method of claim 1, wherein the vesicles are selected from the group consisting of exosomes, microparticles, microvesicles, nanosomes, extracellular vesicles, and ectosomes.

5. A composition for isolating brain-derived vesicles, the composition comprising an agent for measuring an expression level of at least one selected from the group consisting of CADM2, APLP1, EPHA7, CRLF1, and SLC6A3 in a biological sample including vesicles.

6. The composition of claim 5, wherein the biological sample is a cell culture supernatant, whole blood, serum, plasma, ascites fluid, cerebrospinal fluid, bone marrow aspirate, bronchoalveolar lavage fluid, urine, semen, vaginal fluid, mucus, saliva, sputum, or purified lysates from a biological tissue sample.

7. The composition of claim 5, wherein the brain-derived vesicles are neuron-derived vesicles, oligodendrocyte-derived vesicles, microglia-derived vesicles, astrocyte-derived vesicles, or a combination thereof.

8. The composition of claim 5, wherein the vesicles are selected from the group consisting of exosomes, microparticles, microvesicles, nanosomes, extracellular vesicles, and ectosomes.

9. The composition of claim 5, wherein the agent is an antibody or antigen-binding fragment thereof specifically binding to at least one selected from the group consisting of CADM2, APLP1, EPHA7, CRLF1, and SLC6A3 or a fragment thereof.

10. A method of providing information for diagnosis of a degenerative brain disease, the method comprising:
obtaining a biological sample including vesicles from a subject;
isolating brain-derived vesicles by analyzing an expression level of at least one selected from the group consisting of CADM2, APLP1, EPHA7, CRLF1, and SLC6A3 in the biological sample; and
comparing the expression levels of CADM2, APLP1, EPHA7, CRLF1, and SLC6A3 measured in the brain-derived vesicles with an expression level of a control.

11. The method of claim 10, wherein the degenerative brain disease is selected from the group consisting of Alzheimer's disease, Parkinson's disease, Huntington's disease, stroke, multiple system atrophy, vascular disease dementia, frontotemporal dementia (FTD), cortical basal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron disease, Guam parkinsonism-dementia complex, FTDP-17, Lytico-Bodig disease, multiple sclerosis, and traumatic brain injury (TBI).

12. A composition for diagnosing a degenerative brain disease, the composition comprising an agent for measuring an expression level of at least one selected from the group consisting of CADM2, APLP1, EPHA7, CRLF1, and SLC6A3 in a biological sample including vesicles.


SEQUENCE LISTING (6 sequences)

SEQ ID NO: 1 - DNA, 20 bp, Artificial
  Aplp1 F primer
gccactgtcattgctgcttc

SEQ ID NO: 2 - DNA, 21 bp, Artificial
  Aplp1 R primer
gggttagacgcccacatagtc

SEQ ID NO: 3 - DNA, 22 bp, Artificial
  Cadm2 F primer
cgatccgattgtcccttaaagc

SEQ ID NO: 4 - DNA, 21 bp, Artificial
  Cadm2 R primer
agggcagtttccactaactca

SEQ ID NO: 5 - DNA, 21 bp, Artificial
  L1CAM F primer
aaaggtgcaagggtgacattc

SEQ ID NO: 6 - DNA, 19 bp, Artificial
  L1CAM R primer
tccccacgttcctgtaggt